(19) United States
(12) Patent Application Publication
(10) Pub. No.: US 2005/0240120 A1
(43) Pub. Date: Oct. 27, 2005

(54) Vise and method of use

(76) Inventor: D. Bruce Modesitt, San Carlos, CA (US)

Correspondence Address: HAYNES BEFFEL & WOLFELD LLP, P O BOX 366, HALF MOON BAY, CA, 94019, US

(21) Appl. No.: 10/831,852

(22) Filed: Apr. 26, 2004

(51) Int. Cl. A61B 5/00; A61M 25/00; A61M 25/09; A61M 25/082; A61M 25/098

(52) U.S. Cl. 600/585; 604/528

(57) ABSTRACT

A device for releasably attaching to an elongated element is disclosed. Examples of the elongated element include a shaft, such as a guidewire. The device can improve the ergonomics of manipulating the elongated element. The device can also be easily put onto and removed from the guidewire. The invention also relates to a method of using the device.

### BACKGROUND OF THE INVENTION

1. Field of the Invention

The present invention relates generally to a device for releasably attaching to an elongated element, for example a shaft such as a guidewire. The invention also relates to a method of using the device.

2. Description of the Related Art

During minimally invasive vascular surgery, a long guidewire is often inserted into the vessel. The surgeon controls movement of the portion of the guidewire inside the vessel by physically manipulating the portion of the guidewire that is outside of the patient's body. The guidewire is also often withdrawn and redeployed entirely from the body.

The guidewires are difficult to precisely manipulate without a handle, but the handle must be able to controllably slide along the length of the guidewire as the guidewire is being insert and removed from the body. The handle must also be able to be removed from and reattached to the guidewire entirely when the guidewire is removed from the body.

However, the time spent manipulating the handle in the operating room should be minimized. The less steps needed to move the handle relative to the guidewire, the better. Guidewire handles are often therefore configured as pin vises. Pin vises can grab and release the guidewire with a minimal number of steps.

Many pin vises slide over the end of guidewire. U.S. Pat. No. 4,858, 810 by Intelkofer et al. and U.S. Pat. Nos. 5,159,861 and 5,325,746 by Anderson teach examples of such a pin vise. These pin vises often provide an ergonomic handle with which the surgeon can hold the guidewire. These pin vises also often enable the surgeon to grasp and release the guidewire from the pin vise using only one hand.

To remove theses pin vises from the guidewire, however, the pin vises must be slid down the entire length of the guidewire until the pin vise comes off the open end of the guidewire. These pin vises can be very time consuming and unergonomic to put on and/or remove from the guidewire. These pin vises also often take two hands to put on and/or remove from the guidewire, as one hand steadies the guidewire as the other hand moves the pin vise.

VascuMetrix, LLC (Mesa, Ariz.) manufactures a pin vise that is made from a threaded collet rotatably mounted on a threaded three-jaw chuck. The collet and the chuck have side ports that access an inner channel. During use, the guidewire is fed through the ports and into the inner channel. The collet is then be screwed down onto the chuck and the jaws of the chuck are deformably closed onto the guidewire, thereby grasping the guidewire with the pin vise.

The VascuMetrix pin vise can be operated with a single hand. The pin vise can also be put on and/or removed from the guidewire, via the side port, without sliding the pin vise to the open end of the guidewire. However, when the VascuMetrix pin vise is grasping the guidewire, the grasping force is applied over a small area where the jaws of the tips of the chuck are deformed inward. The pressure applied on the guidewire is therefore very large and can easily damage the delicate guidewire. Furthermore, there is no safety mechanism on the VascuMetrix pin vise to prevent over-tightening the pin vise onto, and thereby damaging, the guidewire. The collet also must be rotated numerous times to sufficiently grasp and release the guidewire.

Therefore there exists a need for a pin vise and method of use that reduces pressure applied to a guidewire when held. There also exists a need for a pin vise that can engage the guidewire wire from side, or otherwise provide a quick release from, and attachment to, the guidewire. Furthermore, a need exists to have a pin vise that also has improved ergonomics, such as being usable by single hand. Also, a need exists for a pin vise with a reduced amount of time needed to grasp and release the guidewire. There is also a need to have a pin vise that fulfills the above needs and also prevents overcrimping the guidewire.

### BRIEF SUMMARY OF THE INVENTION

A vise for manipulating an intravascular leader is disclosed. The vise has a body. The body has a channel, a first longitudinal end, a second longitudinal end, and an external port. The external port provides access to the channel. The external port extends from the first longitudinal end to the second longitudinal end when the vise is in a first configuration. The channel has a first minimum channel diameter when the vise is in a first configuration. The channel can grasp the intravascular leader when the vise is in a second configuration.

The body can have a handle and a cam. The handle can be rotatably attached to the cam. The handle can be eccentrically rotatably attached to the cam. The handle can have a first portion of the channel. The cam can have a second portion of the channel.

When the vise is in the second configuration, the handle can be eccentrically rotated with respect to the cam such that the channel has a second minimum channel diameter. The second minimum channel diameter can be less than the first minimum channel diameter.

The cam can have a first cam part and a second cam part. The first cam part can be configured to move toward the second cam part when the vise is moved from the first configuration to the second configuration.

Another vise is disclosed for manipulating an intravascular leader. The vise has a body that has a first body section rotatably attached to a second body section. The body also has a first longitudinal end, a second longitudinal end, and an external port. The first body section has a first channel. The second body section has a second channel. The first channel is eccentrically aligned with the second channel. The external port provides access to the first and second channels. The external port extends from the first longitudinal end to the second longitudinal end when the vise is in a first configuration.

A method of applying a force to an intravascular leader with a vise is also disclosed. The vise has a channel, a first vise end, a second vise end, and a side access port. The method includes inserting the intravascular leader into the side port of the vise. The method also includes rotating the first vise end with respect to the second vise end. Rotating causes the channel to grasp the intravascular leader. The method further includes applying a force to the vise.

The channel can have a first channel and a second channel. Rotating the first vise end with respect to the second vise end can also include misaligning the first channel with respect to the second channel. Misaligning the first channel with respect to the second channel can include offsetting the first channel with respect to the second channel. Misaligning can include moving the first channel to not be parallel with respect to the second channel.

### BRIEF DESCRIPTION OF THE DRAWINGS

FIG. 1 illustrates an embodiment of the vise.

FIG. 2 is a perspective view of an embodiment of the first section of the vise of FIG. 1.

FIG. 3 is an end view of the first section of FIG. 2.

FIG. 4 is a perspective view of an embodiment of the second section of the vise of FIG. 1.

FIG. 5 is an end view of the second section of FIG. 4.

FIG. 6 illustrates an embodiment of the vise.

FIG. 7 illustrates section A-A of FIG. 6.

FIG. 8 illustrates an embodiment of the vise.

FIG. 9 illustrates an embodiment of section B-B of FIG. 8.

FIG. 10 is a front end view of an embodiment of the first section of FIG. 8.

FIG. 11 is a perspective view of the first section of FIG. 8.

FIG. 12 is a perspective view of the second section of FIG. 8.

FIG. 13 is a front end view of an embodiment of the cam of FIG. 8.

FIG. 14 is a side perspective exploded view of the cam of FIG. 13.

FIGS. 15, 17 and 19 illustrate an embodiment of a method of using the vise of FIG. 1.

FIGS. 16, 18, 20 and 21 illustrate embodiments of sections C-C, D-D, E-E and F-F respectively.

FIGS. 22 and 23 illustrate section A-A of an embodiment of a method of using the vise of FIG. 6

FIGS. 24 and 26 illustrate an embodiment of a method of using the vise of FIG. 8.

FIGS. 25 and 27 illustrate embodiments of sections G-G and H-H, respectively.

### DETAILED DESCRIPTION

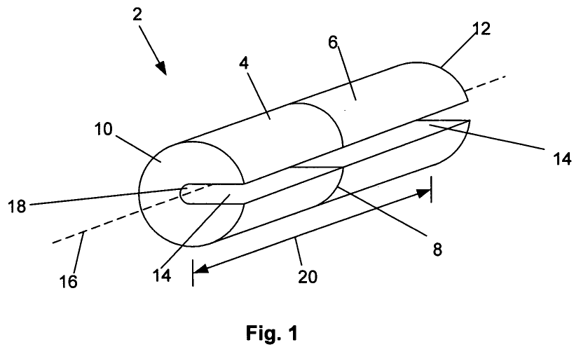

FIG. 1 illustrates a releasable attachment device, for example a vise body 2. The vise body 2 can have a first section 4 and a second section 6. The sections 4 and 6 can have an exterior that can be shaped ergonomically suitable to be handles. The first section 4 can be rotatably attached to the second section 6, for example, at a joint 8. The first section 4 can have a first end 10 away from the joint 8. The second section 6 can have a second end 12 away from the joint 8.

The vise body 2 can have a port 14. The port 14 can be parallel to a longitudinal axis 16 of the vise body 2. The port 14 can access a channel 18. The channel 18 can be parallel to the longitudinal axis 16. The channel 18 can extend from the first end 10 to the second end 12. The port 14 can extend radially from the channel 18 to the exterior of the vise body 2.

The vise body 2 can have a vise length 20 substantially parallel to the longitudinal axis 16. The vise length 20 can be from about 3.8 cm (1.5 in.) to about 15 cm (6.0 in.), for example about 6.1 cm (2.4 in.), also for example about 6.4 cm (2.5 in.). The port 14 can extend longitudinally along substantially all or most of the vise length 20.

Any or all elements of the vise body 2 can be made from, for example, a single or multiple stainless steel alloys, nickel titanium alloys (e.g., Nitinol), cobalt-chrome alloys (e.g., ELGILOY® from Elgin Specialty Metals, Elgin, Ill.; CONICHROME® from Carpenter Metals Corp., Wyomissing, Pa.), molybdenum alloys (e.g., molybdenum TZM alloy, for example as disclosed in International Pub. No. WO 03/082363 A2, published Oct. 9, 2003, which is herein incorporated by reference in its entirety), tungsten-rhenium alloys, for example, as disclosed in International Pub. No. WO 03/082363, polymers such as polyester (e.g., DACRON® from E. I. Du Pont de Nemours and Company, Wilmington, Del.), polypropylene, polytetrafluoroethylene (PTFE), expanded PTFE (ePTFE), polyether ether ketone (PEEK), nylon, polyether-block co-polyamide polymers (e.g., PEBAX® from ATOFINA, Paris, France), aliphatic polyether polyurethanes (e.g., TECOFLEX® from Thermedics Polymer Products, Wilmington, Mass.), polyvinyl chloride (PVC), polyurethane, thermoplastic, fluorinated ethylene propylene (FEP), extruded collagen, silicone, or combinations thereof.

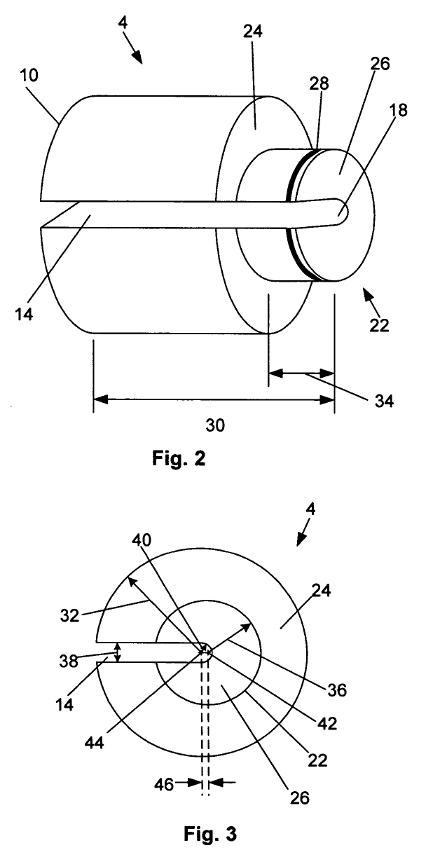

FIGS. 2 and 3 illustrate that the first section 4 of the vise body 2 can have a cam 22. The cam 22 can extend from a face 24 on the first section 4 that can form part of the joint 8 (shown in FIG. 1). The cam 22 can extend to a cam end 26. The cam 22 can be substantially cylindrically shaped. The cam 22 can be a separate element fixedly attached to the first section 4.

The cam 22 can be an integral with the first section 4. The cam 22 can have a first joint engager 28, for example a lip, tab, peg, pin, groove, rail, track, magnet or combination thereof. The first joint engager 28 can substantially circumscribe the cam 22. The first joint engager 28 can partially circumscribe the cam 22.

The first section 4 can have a first section length 30 and a first section radius 32. The first section length 30 can be from about 1.9 cm (0.75 in.) to about 7.6 cm (3.0 in.), for example 3.0 cm (1.2 in.), also for example about 3.3 cm (1.3 in.). The first section radius 32 can be from about 2.5 mm (0.10 in.) to about 4.45 mm (1.75 in.), for example about 3.97 mm (0.1563 in.), also for example about 5.1 mm (0.20 in.).

The cam 22 can have a cam length 34 and a cam radius 36. The cam length 34 can be from about 2.0 mm (0.080 in.) to about 51 mm (2.0 in.), for example about 5.1 mm (0.20 in.), also for example about 4.1 mm (0.16 in.). The cam radius 36 can be from about 1.1 mm (0.045 in.) to about 38 mm (1.5 in.), for example about 2.381 mm (0.09375 in.), also for example about 3.43 mm (0.135 in.).

The port 14 can have a port height 38. The port height 38 can be greater than or about equal to the diameter of a guidewire to be grasped by the vise body 2. The port height 38 can be from about 0.36 mm (0.014 in.) to about 1.1 mm (0.045 in.), for example about 0.993 mm (0.0391 in.), also for example about 1.1 mm (0.045 in.).

The channel can have a channel radius 40. The channel radius 40 can be less than, greater than, or about equal to the port height 38. The channel radius 40 can be about equal to the diameter of the guidewire to be grasped by the vise body 2. The channel radius 40 can be from about 0.36 mm (0.014 in.) to about 1.1 mm (0.045 in.), for example about 0.993 mm (0.0391 in.), also for example about 1.1 mm (0.045 in.).

The cam 22 can have a cam center 42. The cam center 42 can be the center of rotation of the cam 22 during use. The channel 18 can have a channel center 42. In the first section 4, the channel center 42 can be a first offset 46 distance away from the cam center 42. The first offset 46 can be from about 0.03 mm (0.001 in.) to about 1.3 mm (0.050 in.), for example about 0.13 mm (0.0050 in.).

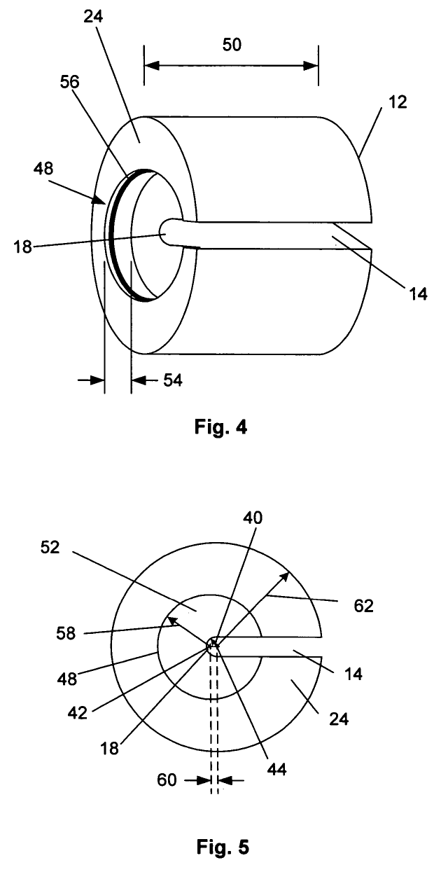

FIGS. 4 and 5 illustrate that the second section 6 can have a cam receptacle 48. The second section 6 can have a second section length 50 from about 1.9 cm (0.75 in.) to about 7.6 cm (3.0 in.), for example about 3.0 cm (1.2 in.), also for example about 3.3 cm (1.3 in.).

The cam receptacle 48 can be configured to rotatably attach to the cam 22. The cam receptacle 48 can recess from the face 24 on the second section 6. The face 24 on the second section 6 can form part of the joint 8 (shown in FIG. 1).

The cam receptacle 48 can extend to a cam receptacle end 52. The cam receptacle 48 can be substantially cylindrically shaped. The cam receptacle 48 can have a cam receptacle length 54. The cam receptacle length 54 can be greater than or about to the cam length 34. The cam receptacle length 54 can be from about 2.0 mm (0.080 in.) to about 51 mm (2.0 in.), for example about 5.1 mm (0.20 in.), also for example about 4.1 mm (0.16 in.).

The cam receptacle 48 can have a second joint engager 56. The second joint engager 56 can be configured to slidably engage the first joint engager 28. The second joint engager 56 can substantially circumscribe the cam receptacle 48. The second joint engager 56 can partially circumscribe the cam receptacle 48.

The cam receptacle 48 can have a cam receptacle radius 58. The cam receptacle radius 58 can be greater than or about equal to the cam radius 36. The cam receptacle radius 58 can be from about 1.1 mm (0.045 in.) to about 38 mm (1.5 in.), for example about 2.381 mm (0.09375 in.), also for example about 3.43 mm (0.135 in.).

In the second section 6, the channel center 42 can be a second offset 60 distance away from the cam center 42. The second offset 60 can be about equal to the first offset 46. The second offset 60 can be from about 0.03 mm (0.001 in.) to about 1.3 mm (0.050 in.), for example about 0.13 mm (0.0050 in.).

The second section 6 can have a second section radius 62. The second section radius 62 can be about equal to the first section radius 32. The second section radius 62 can be from about 2.5 mm (0.10 in.) to about 4.45 mm (1.75 in.), for example about 3.97 mm (0.1563 in.), also for example about 5.1 mm (0.20 in.).

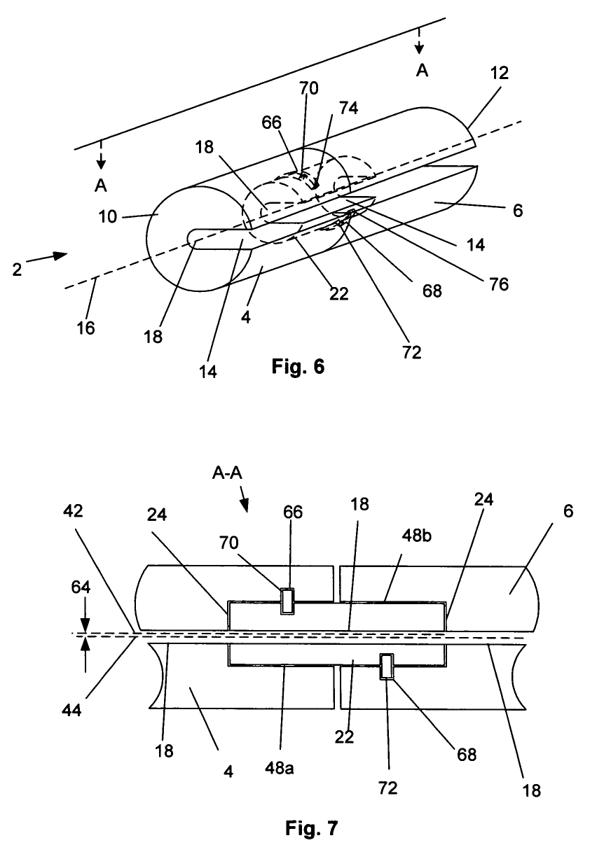

FIGS. 6 and 7 illustrate that the vise body 2 can have a distinct cam 22 that can be rotatably attached to the first section 4 and the second section 6. The first section 4 can have a first cam receptacle 48a. The second section 6 can have a second cam receptacle 48b. The cam center 42 can have an offset 64 from the channel center 42. The offset 64 can be from about 0.03 mm (0.001 in.) to about 1.3 mm (0.050 in.), for example about 0.13 mm (0.0050 in.).

A first stop track 66 can be in the first cam receptacle 48a (as shown) or in the cam 22. The first stop track 66 can partially circumscribe the first cam receptacle 48a or the cam 22. A second stop track 68 can be in the second cam receptacle 48b (as shown) or in the cam 22. The second stop track 68 can partially circumscribe the second cam receptacle 48b or the cam 22. The first stop track 66 can be angularly offset from the second stop track 68 with respect to the longitudinal axis 16.

A first stop pin 70 can be fixedly attached or integral to the first cam receptacle 48a or the cam 22, whichever does not have the first stop track 66. The first stop pin 70 can be slidably attached to the first stop track 66.

A second stop pin 72 can be fixedly attached or integral to the second cam receptacle 48b or the cam 22, whichever does not have the second stop track 68. The second stop pin 72 can be slidably attached to the second stop track 68.

The first stop track 66 can have a first stop 74. The second stop track 68 can have a second stop 76. The first and/or second stops 74 and/or 76 can be configured to magnetically fix, snap or otherwise interference fit the first and/or second stop pins 74 and/or 76, respectively.

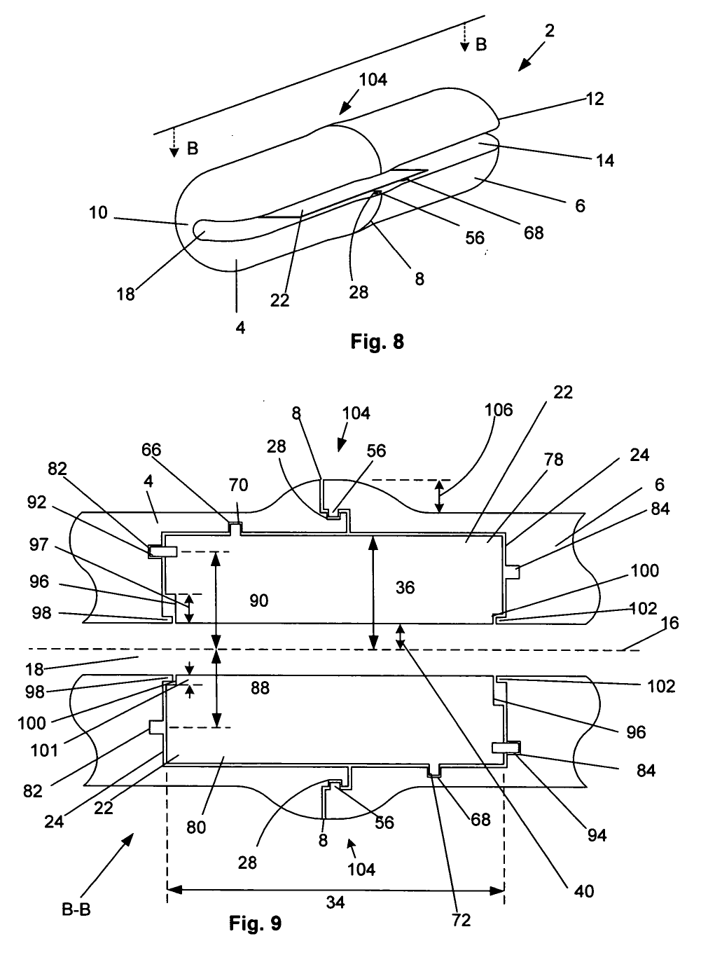

FIGS. 8 and 9 illustrate that the vise body 2 can have the channel radius 40 and/or the channel center 42 that can be variably adjustable over the length of the cam 22. The cam 22 can have a first part 78. The cam 22 can have a second part 80. The first part 78 can be slidably attached to the second part 80. The first part 78 can be configured to move towards and away from the second part 80.

A first crimp track 82 can be in the first cam receptacle 48a (as shown) or in the cam 22. The first crimp track 82 can partially circumscribe the first cam receptacle 48a or the cam 22. A second crimp track 84 can be in the second cam receptacle 48b (as shown) or in the cam 22. The second crimp track 84 can partially circumscribe the second cam receptacle 48b or the cam 22. The first crimp track 82 can be angularly offset from the second crimp track 84 with respect to the longitudinal axis 16.

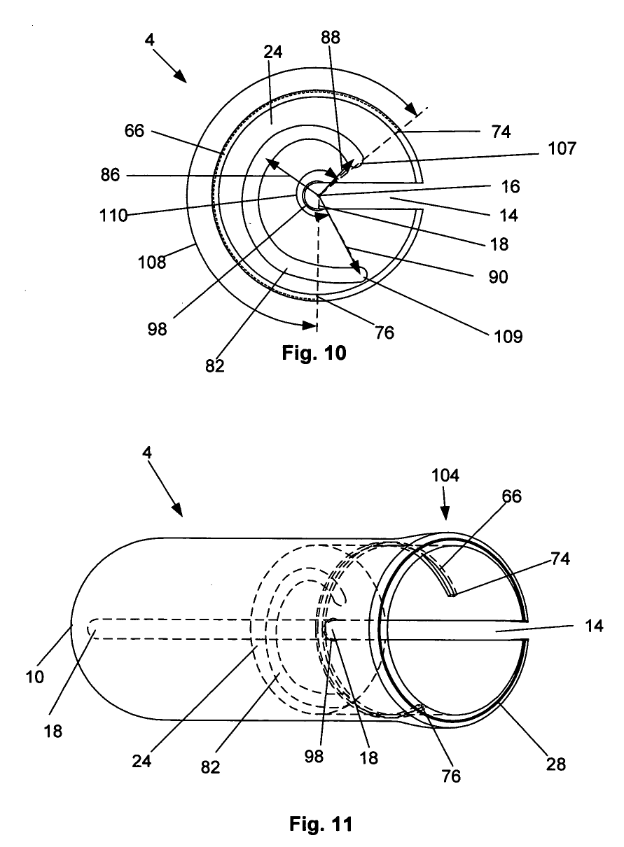

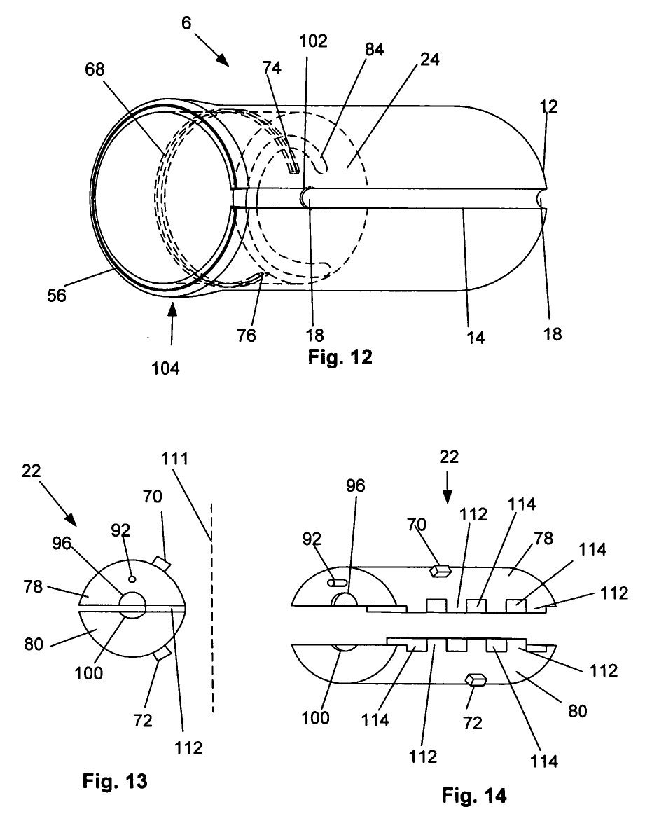

As shown in FIGS. 9 through 12, the first and second crimp tracks 82 and 84 can have crimp track radii 86. The crimp track radii 86 can vary between minimum crimp track radii 88 and maximum crimp track radii 90 with respect to the angle of the crimp track 110 about the longitudinal axis 16.

A first crimp pin 92 can be fixedly attached or integral to the first cam receptacle 48a or the cam 22, whichever does not have the first crimp track 82. The first crimp pin 92 can be slidably and/or rotatably attached to the first crimp track 82.

A second crimp pin 94 can be fixedly attached or integral to the second cam receptacle 48b or the cam 22, whichever does not have the second crimp track 84. The second crimp pin 94 can be slidably and/or rotatably attached to the second crimp track 84.

A first hub engagement 96 can be in the first cam receptacle 48a or the cam 22 (as shown). A first hub 98 can be fixedly attached to or integral with the first cam receptacle 48a (as shown) or the cam 22, whichever does not have the first hub engagement 96. The first hub 98 can be configured to slidably and/or rotatably attach to the first hub engagement 96.

A second hub engagement 100 can be in the second cam receptacle 48b or the cam 22 (as shown). A second hub 102 can be fixedly attached to or integral with the second cam receptacle 48b (as shown) or the cam 22, whichever does not have the second hub engagement 100. The second hub 102 can be configured to slidably and/or rotatably attach to the second hub engagement 100.

The first and second hub engagements 96 and 100 can have first and second hub engagement heights 97 and 101. The first hub engagement height 97 minus the second hub engagement height 101 can be greater than of about equal to the maximum crimp track radius 90 minus the minimum crimp track radius 88. The first hub engagement height 97 can be from about 0.51 mm (0.020 in.) to about 38 mm (1.5 in.), for example about 1.5 mm (0.060 in.). The second hub engagement height 101 can be from about 0.25 mm (0.010 in.) to about 38 mm (1.5 in.), for example about 1.0 mm (0.040 in.).

The vise body 2 can have a grip ridge 104 at and/or around the joint 8. The grip ridge 104 can be configured to be ergonomically beneficial for gripping with a hand. The grip ridge 104 can have a grip ridge height 106. The grip ridge height 106 can be from about 0 mm (0 in.) to about 4.3 cm (1.7 in.), for example about 1.4 mm (0.055 in.).

The first section 4 can have a first joint engager 28. The second section 6 can have a second joint engager 56. The first joint engager 28 can be configured to rotatably and/or slidably attach to the second joint engager 56. The first joint engager 28 can form a snap and/or interference fit with the second joint engager 56 in the direction parallel to the longitudinal axis 16.

The vise body 2 can have the first and/or second stop tracks 66 and/or 68, and first and/or second stop pins 70 and/or 72, as described supra. A stop track angle 108 can be the angle about the longitudinal axis 16 from the first stop 74 to the second stop 76. The stop track angle 108 can be from about 10° to about 355°, for example about 24°.

A crimp track angle 110 can be the angle about the longitudinal axis 16 from a crimp track first end 107 to a crimp track second end 109. The crimp track angle 110 can be from about 10° to about 505°, for example about 235°.

FIGS. 13 and 14 illustrate that the first part 78 can be configured to slidably attach to the second part 80, for example parallel to a transverse axis 111. The first and/or second parts 78 and/or 80 can have teeth 112. The first and/or second parts 78 and/or 80 can have teeth receptacles 114. The teeth 112 on one part 78 or 80 can be configured to slidably engage and attach to the teeth receptacles 114 on the opposing part 80 or 78.

Method of Manufacture

Appropriate elements of the vise body 2 can be directly attached by, for example, melting, screwing, gluing, welding or use of an interference fit or pressure fit such as crimping, or combining methods thereof. Appropriate elements, for example the first section 4 and the second section 6, can be snap fit. Appropriate elements can be integrated, for example, molding, die cutting, laser cutting, electrical discharge machining (EDM) or stamping from a single piece or material. Any other methods can be used as known to those having ordinary skill in the art.

Method of Use

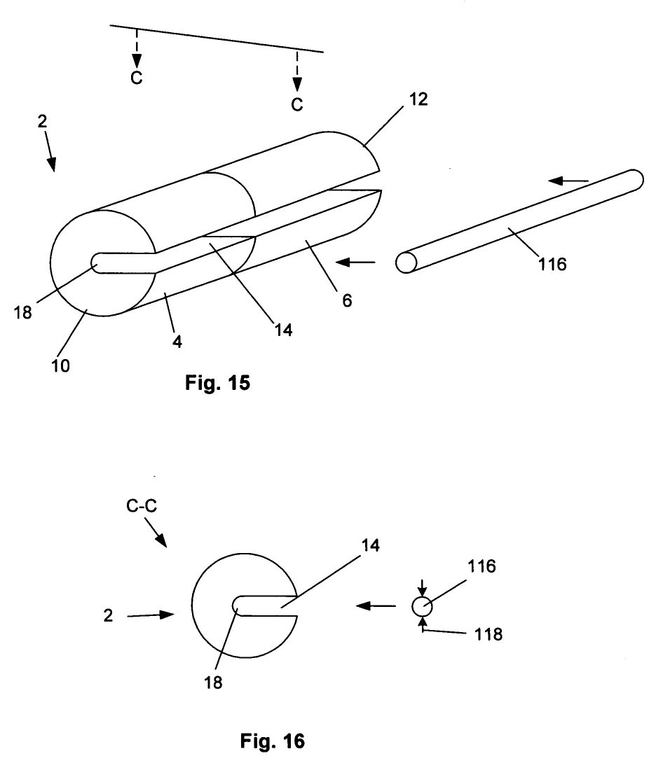

FIGS. 15 through 23 illustrate various methods of using the vise body 2. FIGS. 15 and 16 illustrate a method of moving, as shown by arrows, an elongated leader, such as a guidewire 116, into the vise body 2 when the vise body 2 is in an open configuration. The vise body 2 can be in an open configuration when the port 14 of the first section 4 is aligned with the port 14 of the second section 6. The entire channel 18 can be easily accessed by the guidewire 116 when the vise body 2 is in an open configuration.

The guidewire 116 can have a guidewire diameter 118. The guidewire diameter 118 can be from about 0.36 mm (0.014 in.) to about 1.1 mm (0.045 in.), for example about 1.0 mm (0.040 in.).

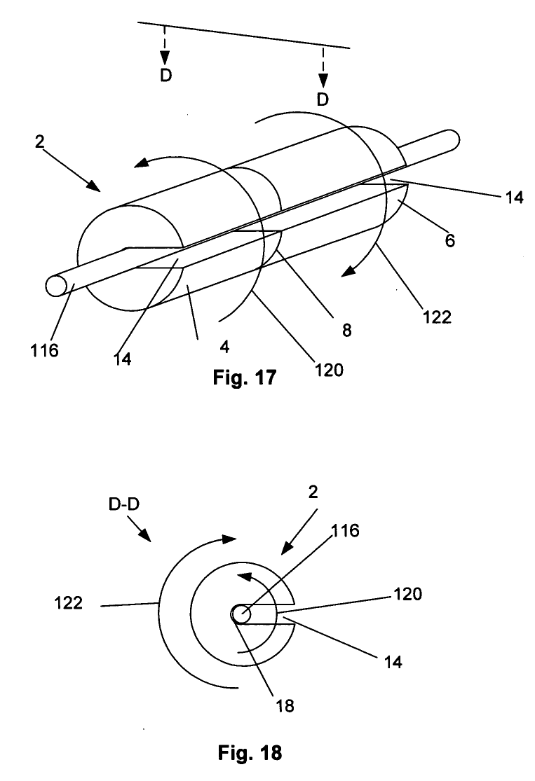

FIGS. 17 and 18 illustrate that once the guidewire 116 is placed in the channel 18, the first section 4 can be rotated, as shown by arrow 120, with respect to second section 6. The second section 6 can be held fixed or rotated, as shown by arrow 122, in the opposite direction of the rotation, as shown by arrow 120, of the first section 4.

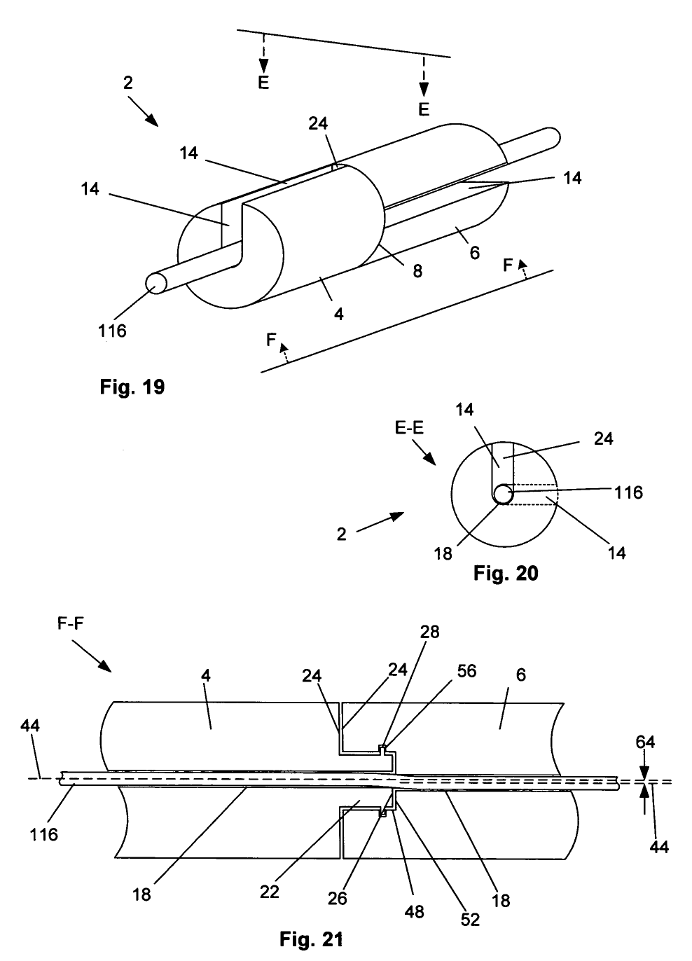

FIGS. 19 and 21 illustrate the vise body 2 and guidewire 116 that can be graspingly configured after being subjected to the rotation and possible counter-rotation shown in FIGS. 17 and 18. The guidewire 116 can be grasped and fixedly attached to the vise body 2.

The port 14 of the first section 4 can be misaligned with the port 14 of the second section 6. The extended axis of the channel center 42 of the first section 4 can be the offset 64 distance from the channel center 42 of the second section 6, as shown in FIG. 21. The channel 18 can grasp the guidewire 116 by applying force to the guidewire 116 around the cam end 26 and the cam receptacle end 52. The guidewire 116 can be released from the vise body 2 by rotating the first section 4 and possibly the second section 6 in the opposite directions from those shown in FIG. 17.

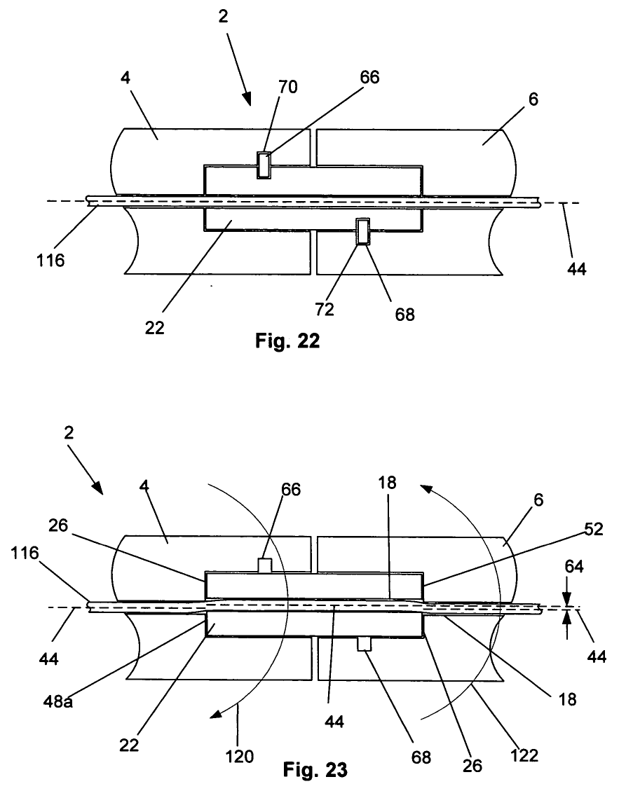

FIG. 22 illustrates the guidewire 116 that can be loaded into the vise body 2 that can have the cam 22 that can be distinct from the first or second sections 4 or 6. FIG. 23 illustrates that the first section 4 can be rotated, as shown (out of plane) by arrow 120. The second section 6 can be held fixed or rotated, as shown (out of plane) by arrow 122, in the opposite direction of the rotation, as shown (out of plane) by arrow 120, of the first section 4. Rotation of the first section 4 and possibly the second section 6 can drive the cam 22 to rotate—or the cam 22 can remain stationary as the first section 4 and possibly the second section 6 rotate around the cam 22.

When the vise body 2 is in the grasping configuration as shown in FIG. 23, the extended axis of the channel center 42 in the cam 22 can be the offset 64 distance from the channel center 42 in the first and second sections 4 and 6. The channel 18 can grasp the guidewire 116 by applying force to the guidewire 116 distributed around the area of the cam ends 26 and the cam receptacle ends 52. The guidewire 116 can be released from the vise body 2 by rotating the first section 4 and possibly the second section 6 in the opposite directions from those directions shown in FIG. 23.

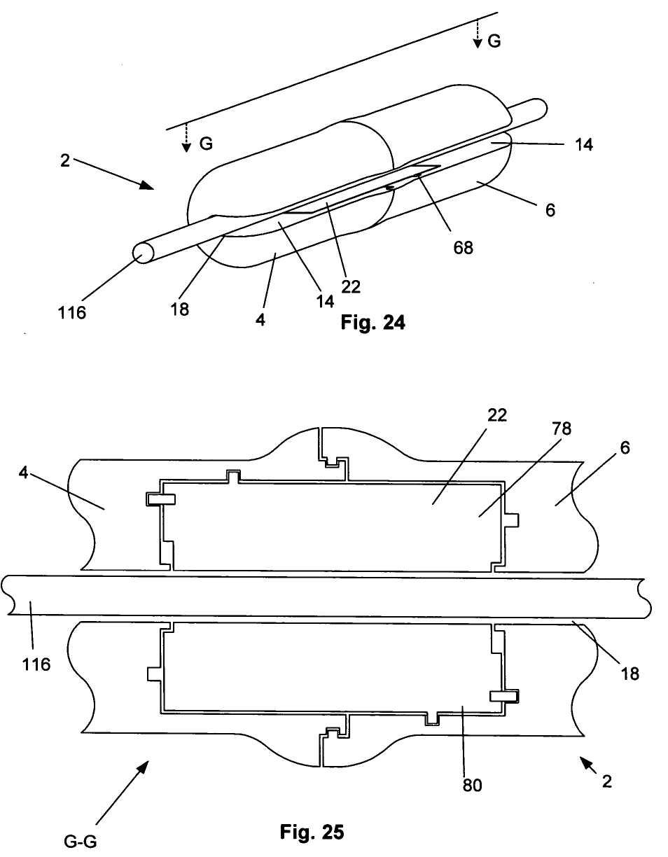

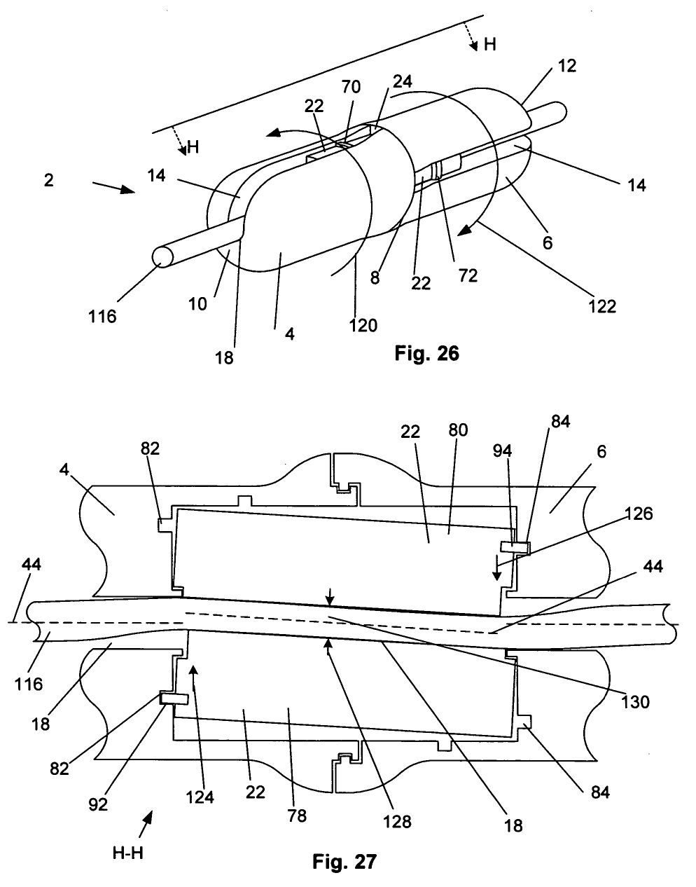

FIGS. 24 and 25 illustrate the guidewire 116 that can be loaded into the vise body 2 that can have the cam 22 that can be configured to variably adjust the channel radius and/or the channel center 42 over the length of the cam 22. FIGS. 26 and 27 illustrate that the first section 4 can be rotated, as shown by arrow 120. The second section 6 can be held fixed or rotated, as shown by arrow 122, in the opposite direction of the rotation, as shown by arrow 120, of the first section 4.

Rotation of the first section 4 and possibly the second section 6 can drive the 23 cam 22 to rotate—or the cam 22 can remain stationary as the first section 4 and possibly the second section 6 rotate around the cam 22. As the cam 22 is rotated with respect to the first and/or second sections 4 and/or 6, the crimp pins 92 and 94 can slide along the crimp tracks 82 and 84.

As the crimp pins 92 and 94 slide along the crimp tracks 82 and 84, the end of the first part 78 of the cam 22 nearest the first crimp pin 92 can be shifted, as shown by arrow 124, into the channel 18, and the end of the second part 80 of the cam 22 nearest the second crimp pin 94 can be shifted, as shown by arrow 126, into the channel 18. The first hub engagements 96 can be guided, in contact or in proximity, by the hubs 98 and 102.

The first part 78 can be brought closer to the second part 80, as shown by arrows 128. The portion of the channel 18 that passes through the cam 22 can have a crimped channel radius 130. The crimped channel radius 130 can be less than or about equal to the channel radius 40. The crimped channel radius 130 can be from about 0.2 mm (0.007 in.) to about 1.1 mm (0.045 in.), for example about 0.76 mm (0.030 in.). The channel center 42 in the cam 22 can be unparallel with the channel center 42 outside of the cam 22.

The force grasping the guidewire 116 exerted by the vise body 2 can by applied across all or part the area of the surface of the cam 22 along the channel 18, and possibly across the area of the surface of the first and/or second sections 4 and/or 6 along the channel 18 adjacent to the cam 22. The guidewire 116 can be released from the vise body 2 by rotating the first section 4 and possibly the second section 6 in the opposite directions from those directions shown in FIG. 26.

It is apparent to one skilled in the art that various changes and modifications can be made to this disclosure, and equivalents employed, without departing from the spirit and scope of the invention. Elements shown with any embodiment are exemplary for the specific embodiment and can be used on other embodiments within this disclosure.

1. A vise for manipulating an intravascular leader, the vise comprising:
a body comprising a channel, a first longitudinal end, a second longitudinal end, and an external port, and
wherein the external port provides access to the channel, and wherein the external port extends from the first longitudinal end to the second longitudinal end when the vise is in a first configuration, and wherein the channel has a first minimum channel diameter when the vise is in a first configuration, and wherein the channel can grasp the intravascular leader when the vise is in a second configuration.

2. The vise of claim 1, wherein the body comprises a handle and a cam, and wherein the handle is rotatably attached to the cam.

3. The vise of claim 2, wherein the handle is eccentrically rotatably attached to the cam.

4. The vise of claim 2, wherein the handle comprises a first portion of the channel.

5. The vise of claim 4, wherein the cam comprises a second portion of the channel.

6. The vise of claim 1, wherein when the vise is in the second configuration, the handle is eccentrically rotated with respect to the cam such that the channel has a second minimum channel diameter, wherein the second minimum channel diameter is less than the first minimum channel diameter.

7. The vise of claim 1, further comprising a cam, wherein the cam comprises a first cam part and a second cam part.

8. The vise of claim 7, wherein the first cam part is configured to move toward the second cam part when the vise is moved from the first configuration to the second configuration.

9. The vise of claim 1, wherein the body comprises a first section and a second section, and wherein the vise further comprises a cam.

10. The vise of claim 9, wherein the first section comprises a first vise end, and wherein the second section comprises a second vise end, and wherein the first vise end is rotatably attached to the second vise end, and wherein the cam is rotatably attached to the first vise end.

11. The vise of claim 10, wherein the cam is eccentrically rotatably attached to the first vise end.

12. The vise of claim 11, wherein the cam is rotatably attached to the second vise end.

13. The vise of claim 12, wherein the cam is eccentrically rotatably attached to the second vise end.

14. The vise of claim 1, wherein the body further comprises a first section and a second section, and wherein the vise is moved from the first configuration to the second configuration by rotating the first section with respect to the second section.

15. The vise of claim 1, further comprising a longitudinal axis, wherein the port extends radially from the longitudinal axis.

16. A vise for manipulating an intravascular leader, the vise comprising:
a body a first body section rotatably attached to a second body section, the body further comprising a first longitudinal end, a second longitudinal end, an external port; and
the first body section comprising a first channel; and
the second body section comprising a second channel; and
wherein the first channel is eccentrically aligned with the second channel, and wherein the external port provides access to the first and second channels, and wherein the external port extends from the first longitudinal end to the second longitudinal end when the vise is in a first configuration.

17. A method of applying a force to an intravascular leader with a vise, wherein the vise comprises a channel, a first vise end, a second vise end, and a side access port, the method comprising:
inserting the intravascular leader into the side port of the vise,
rotating the first vise end with respect to the second vise end, wherein rotating causes the channel to grasp the intravascular leader, and
applying a force to the vise.

18. The method of claim 17, wherein the channel comprises a first channel and a second channel, and wherein rotating further comprises misaligning the first channel with respect to the second channel.

19. The method of claim 18, wherein misaligning further comprises offsetting the first channel with respect to the second channel.

20. The method of claim 18, wherein misaligning further comprises moving the first channel to not be parallel with respect to the second channel.
